SEQUENCE LISTING (7 sequences)

SEQ ID NO: 1 - DNA, 1518 bp, Simian immunodeficiency virus
accctctacgtgaccgtgttctacggcgtgccggcctggcgcaacgccacgatcccgctcttctgcgccactaagaaccgcgacacctggggcaccacgcagtgcctcccggacaacggcgactactccgaggtggccctcaacgtgaccgagtccttcgacgcctggaacaataccgtgaccgagcaggctatcgaggacgtgtggcagctcttcgagacctctatcaagccgtgcgtgaagctctccccgctctgcatcacgatgcgctgcaacaagtccgagaccgaccgctggggcctcaccaagtctatcaccacgactgcctccaccacgtccaccacggcctccgccaaggtggatatggtgaacgagacctccagctgcatcgcccaggacaactgcaccggcctcgagcaggagcagatgatctcctgcaagttcaacatgaccggcctcaagcgcgacaagaagaaggagtacaacgagacctggtactccgccgacctcgtgtgcgagcagggcaacaataccggcaacgagtcccgctgctacatgaaccactgcaacacctccgtgatccaggagtcctgcgacaagcactactgggacgctatccgcttccgctactgcgccccgcccggctacgccctcctgcgctgcaacgacaccaactactccggcttcatgccgaagtgctccaaggtggtcgtttccagctgcaccaggatgatggagacccagacctccacctggttcggcttcaacggcacccgcgccgagaaccgcacctacatctactggcacggccgcgacaaccgcacgatcatttccctcaacaagtactacaacctcacgatgaagtgccgcaggccgggcaacaagaccgtgctccccgtgacgatcatgtccggcctcgtgttccactcccagccgatcaacgaccgcccgaagcaggcctggtgctggttcggtggcaagtggaaggacgctatcaaggaggtcaagcagacgatcgtgaagcacccgcgctacaccggcaccaacaataccgacaagatcaacctcaccgccccgggcggtggggacccggaggtcaccttcatgtggaccaactgccgcggcgagttcctctactgcaagatgaactggttcctcaactgggtggaggaccgcaacaccgccaaccagaagccgaaggagcagcacaagcgcaactacgtgccgtgccacatccgccagatcatcaacacctggcacaaggtgggcaagaacgtgtacctcccgccccgcgagggcgacctcacctgcaactccaccgtgacctccctgatcgccaacatcgactggatcgacggcaaccagaccaacatcacgatgtccgccgaggtggccgagctctaccgcctcgagctcggcgactacaagctcgtggagatcaccccgatcggcctcgccccgaccgacgtgaagcgctacactaccggcggtacctcacgcaacaagcgctgatagtaa

SEQ ID NO: 2 - protein, 503 aa, Simian immunodeficiency virus
TLYVTVFYGVPAWRNATIPLFCATKNRDTWGTTQCLPDNGDYSEVALNVTESFDAWNNTVTEQAIEDVWQLFETSIKPCVKLSPLCITMRCNKSETDRWGLTKSITTTASTTSTTASAKVDMVNETSSCIAQDNCTGLEQEQMISCKFNMTGLKRDKKKEYNETWYSADLVCEQGNNTGNESRCYMNHCNTSVIQESCDKHYWDAIRFRYCAPPGYALLRCNDTNYSGFMPKCSKVVVSSCTRMMETQTSTWFGFNGTRAENRTYIYWHGRDNRTIISLNKYYNLTMKCRRPGNKTVLPVTIMSGLVFHSQPINDRPKQAWCWFGGKWKDAIKEVKQTIVKHPRYTGTNNTDKINLTAPGGGDPEVTFMWTNCRGEFLYCKMNWFLNWVEDRNTANQKPKEQHKRNYVPCHIRQIINTWHKVGKNVYLPPREGDLTCNSTVTSLIANIDWIDGNQTNITMSAEVAELYRLELGDYKLVEITPIGLAPTDVKRYTTGGTSRNKR

SEQ ID NO: 3 - DNA, 1961 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgca

SEQ ID NO: 4 - DNA, 1428 bp, Human immunodeficiency virus
gtggagaagctctgggtcaccgtgtactacggcgtgccggtgtggaaggaggccaccaccaccctcttctgcgcctccgacgccaaggcgtacgacaccgaggtgcacaacgtgtgggccacccacgcctgcgtgccgaccgacccgaacccgcaggaggtggtgctggagaacgtgaccgagcacttcaacatgtggaagaacaacatggtggagcagatgcaggaggacatcatctccctctgggaccagtccctcaagccgtgcgtgaagctcaccccgctctgcgtgaccctcaactgcaaggacgtgaacgccaccaacaccaccaacgactccgagggcacgatggagcgcggcgagatcaagaactgctccttcaacatcaccacctccatccgcgacgaggtgcagaaggagtacgccctcttctacaagctcgacgtggtgccgatcgacaacaacaacacctcctaccggttgatctcctgcgacacctccgtgatcacccaggcctgcccgaagatctccttcgagccgatcccgatccactactgcgccccggccggcttcgccatcctcaagtgcaacgacaagaccttcaacggcaagggcccgtgcaagaacgtgtccaccgtgcagtgcacccacggcatccgcccggtggtgtcgacccagctcctcctcaacggctccctcgccgaggaggaggtggtgatccgctccgacaacttcaccaacaacgccaagaccatcatcgtgcagctcaaggagtccgtggagatcaactgcacccgcccgaacaacaacacccgcaagtccatccacatcggcccgggccgcgccttctacaccaccggcgagatcatcggcgacatccgccaggcccactgcaacatctcccgcgccaagtggaacgacaccctcaagcagatcgtgatcaagcttcgcgagcagttcgagaacaagaccatcgtgttcaaccactcgagcggcggcgacccggagatcgtgatgcactccttcaactgcggcggcgagttcttctactgcaactccacccagctcttcaactccacctggaacaacaacaccgagggctccaacaacaccgagggcaacaccatcaccctcccgtgccgcatcaagcagatcatcaacatgtggcaggaggtgggcaaggccatgtacgccccgccgatccgcggccagatccgctgctcctccaacatcaccggcctcctcctcacccgcgacggcggcatcaacgagaacggcaccgagatcttccgcccgggcggcggcgacatgcgcgacaactggcgctccgagctgtacaagtacaaggtggtgaagatcgagcccctaggcgtggccccgaccaaggccaagcgccgcgtggtgcagcgcgagaagcgctgatagtaa

SEQ ID NO: 5 - protein, 473 aa, Human immunodeficiency virus
VEKLWVTVYYGVPVWKEATTTLFCASDAKAYDTEVHNVWATHACVPTDPNPQEVVLENVTEHFNMWKNNMVEQMQEDIISLWDQSLKPCVKLTPLCVTLNCKDVNATNTTNDSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMRDNWRSELYKYKVVKIEPLGVAPTKAKRRVVQREKR

SEQ ID NO: 6 - DNA, 1938 bp, Human immunodeficiency virus
gtggagaagctctgggtcaccgtgtactacggcgtgccggtgtggaaggaggccaccaccaccctcttctgcgcctccgacgccaaggcgtacgacaccgaggtgcacaacgtgtgggccacccacgcctgcgtgccgaccgacccgaacccgcaggaggtggtgctggagaacgtgaccgagcacttcaacatgtggaagaacaacatggtggagcagatgcaggaggacatcatctccctctgggaccagtccctcaagccgtgcgtgaagctcaccccgctctgcgtgaccctcaactgcaaggacgtgaacgccaccaacaccaccaacgactccgagggcacgatggagcgcggcgagatcaagaactgctccttcaacatcaccacctccatccgcgacgaggtgcagaaggagtacgccctcttctacaagctcgacgtggtgccgatcgacaacaacaacacctcctaccggttgatctcctgcgacacctccgtgatcacccaggcctgcccgaagatctccttcgagccgatcccgatccactactgcgccccggccggcttcgccatcctcaagtgcaacgacaagaccttcaacggcaagggcccgtgcaagaacgtgtccaccgtgcagtgcacccacggcatccgcccggtggtgtcgacccagctcctcctcaacggctccctcgccgaggaggaggtggtgatccgctccgacaacttcaccaacaacgccaagaccatcatcgtgcagctcaaggagtccgtggagatcaactgcacccgcccgaacaacaacacccgcaagtccatccacatcggcccgggccgcgccttctacaccaccggcgagatcatcggcgacatccgccaggcccactgcaacatctcccgcgccaagtggaacgacaccctcaagcagatcgtgatcaagcttcgcgagcagttcgagaacaagaccatcgtgttcaaccactcgagcggcggcgacccggagatcgtgatgcactccttcaactgcggcggcgagttcttctactgcaactccacccagctcttcaactccacctggaacaacaacaccgagggctccaacaacaccgagggcaacaccatcaccctcccgtgccgcatcaagcagatcatcaacatgtggcaggaggtgggcaaggccatgtacgccccgccgatccgcggccagatccgctgctcctccaacatcaccggcctcctcctcacccgcgacggcggcatcaacgagaacggcaccgagatcttccgcccgggcggcggcgacatgcgcgacaactggcgctccgagctgtacaagtacaaggtggtgaagatcgagcccctaggcgtggccccgaccaaggccctccgcctgaggctccgcctgaggctccgcctgaggggcatcggcgccgtgttcctcggcttcctcggcgccgccggctccacgatgggcgccgcctccatgaccctcaccgtgcaggcccgcctcctcctctccggcatcgtgcagcagcagaacaacctcctccgcgccatcgaggcccagcagcgcatgctccagctcaccgtgtggggcatcaagcagctccaggcccgcgtgctcgccgtggagcgctacctcggcgaccagcagctcctcggcatctggggctgctccggcaagctcatctgcaccaccgccgtgccgtggaacgcctcctggtccaacaagtccctcgaccgcatctggaacaacatgacctggatggagtgggagcgcgagatcgacaactacacctccgagatctacaccctcatcgaggagtcccagaaccagcaggagaagaacgagcaggagctcctcgagctcgacaagtgggcctccctctggaactggttcgacatcaccaagtggctctggtactgatagtaa

SEQ ID NO: 7 - protein, 643 aa, Human immunodeficiency virus
VEKLWVTVYYGVPVWKEATTTLFCASDAKAYDTEVHNVWATHACVPTDPNPQEVVLENVTEHFNMWKNNMVEQMQEDIISLWDQSLKPCVKLTPLCVTLNCKDVNATNTTNDSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMRDNWRSELYKYKVVKIEPLGVAPTKALRLRLRLRLRLRGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWY

(19) United States
(12) Patent Application Publication
(10) Pub. No.: US 2004/0040061 A1
(43) Pub. Date: Feb. 26, 2004

(54) Expression in plants of HIV-related proteins

(76) Inventors: Michael Horn, College Station, TX (US); Stephen Streatfiled, Bryan, TX (US); Joseph Jilka, College Station, TX (US)

Correspondence Address: Patricia A. Sweeney, 1835 Pleasant St., West Des Moines, IA, 50265-2334, US

(21) Appl. No.: 10/375,657

(22) Filed: Feb. 27, 2003

Related U.S. Application Data
(60) Provisional application No. 60/359,969, filed on Feb. 27, 2002.

(51) Int. Cl. A01H 1/00; C12N 15/82; A01H 5/00

(52) U.S. Cl. 800/288; 800/320.1

(57) ABSTRACT

Plants are engineered to express HIV related surface protein genes. The plants can be used as a source of the protein for a variety of purposes. Plant tissue can be orally administered to animals to elicit an immune response or provide protection from viral infection. The protein can be extracted and delivered to animals. Plant produced proteins can also provide a less expensive and more readily available source of the protein as reagents or in other experimentation involving HIV and SIV proteins.

This application claims priority to previously filed and co-pending application U.S. Serial No. 60/359,969; this application and all references cited herein are incorporated herein by reference.

Work on this invention was funded in part with a grant from the United States Government, the National Institute of Health, Grant No. 1R21A1048374-01, and the Government has certain rights therein.

### BACKGROUND OF THE INVENTION

Over the past decade, transgenic plants have been successfully used to express a variety of genes from bacterial and viral pathogens. Many of the resulting peptides induced an immunogenic response in mice (Mason, H. S., T. A. Haq, J. D. Clements, C. J. Arntzen. 1998. Edible vaccine protects against Escherichia coli heat-labile enterotoxin (LT):potatoes expressing a synthetic LT-B gene. Vaccine 16:13361343; Wigdorovitz, A., C. Carrillo, M. J. Dus Santos, K. Trono, A. Peralta, M. C. Gomez, R. D. Rios, P. M. Franzone, A. M. Sadir, J. M. Escribano, M. V. Borca. 1999. Induction of a protective antibody response to foot and mouth disease virus in mice following oral and parental immunization with alfalfa transgenic plants expressing the viral structural protein VP1. Virology 255:347-353), and humans (Kapusta, J., M. Modelska, M. Figlerowicz, T. Pniewski, M. Letellier, O. Lisowa, V. Yusibov, H. Koprowski, A. Plucienniczak, A. B. Legocki. 1999. A plant-derived edible vaccine against hepatitis B virus. FASEB J. 13:1796-1799) comparable to that of the original pathogen. Characterization studies of these engineered immunogens have proven the ability of plants to express, fold and modify proteins in a manner that is consistent with the native source.

The utilization of transgenic plants for vaccine production has several potential benefits over traditional vaccines. First, transgenic plants are usually constructed to express only a small antigenic portion of the pathogen or toxin, eliminating the possibility of infection or innate toxicity of the whole organism and reducing the potential for adverse reactions. Second, since there are no known human or animal pathogens that are able to infect plants, concerns with viral or prion contamination are eliminated. Third, immunogen production in transgenic crops relies on the same established technologies to sow, harvest, store, transport, and process the plant material as those commonly used for food crops, making transgenic plants a very economical means of large-scale vaccine production. Fourth, expression of immunogens in the natural protein-storage compartments of plant seed maximizes stability, minimizes the need for refrigeration and keeps transportation and storage costs low (Streatfield, S. J., J. M. Jilka, E. E. Hood, D. D. Turner, M. R. Bailey, J. M. Mayor, S. L. Woodard, K. K. Beifuss, M. E. Horn, D. E. Delaney, I. R. Tizard, J. A. Howard. Plant-based vaccines: unique advantages. Vaccine 19:2742-2748; Kapusta, supra). Fifth, formulation of multicomponent vaccines is possible by blending the seed of multiple transgenic corn lines into a single vaccine. Sixth, direct oral administration is possible when immunogens are expressed in commonly consumed food plants, such as grain, leading to the production of edible vaccines.

Numerous genes have been cloned into a variety of transgenic plants including many enzymes that have demonstrated the same enzymatic activity as their authentic counterparts. See, for example, expression of avidin in plants, U.S. Pat. No. 5,767,379; aprotinin expressed in plants, U.S. Pat. No. 5,824,870 and proteases expressed in plants, U.S. Pat. No. 6,087,558.; Hood, E. E., D. R. Withcher, S. Maddock, T. Meyer, C. B. M. Baszczynski, P. Flynn, J. Register, L. Marshal, D. Bond, E. Kulisek, A. Kusnadi, R. Evangelista, Z. Nikolov, C. Wooge, R. J. Mehigh, R. Hernan, W. K. Kappel, D. Ritland, P. C. Li, and J. A. Howard, 1997, Commercial production of avidin from transgenic maize: characterization of transformant, production, processing, extraction and purification. Molecular Breeding 3:291-306; Pen, J., L. Molendijk, W. J. Quax, P. C. Sijmons, A. J. van Ooyen, P. J. van den Elzen, K. Rietveld, and A. Hoekema, 1992, Production of active Bacillus licheniformis α-amylase in tobacco and its application in starch liquefaction. Biotechnology 10:292-296; Trudel, J., C. Potvin, and A. Asselin 1992 Expression of active hen egg white lysozyme in transgenic tobacco. Plant Sci. 87:55-67. Many additional genes have been expressed in plants solely for their immunogenic potential, including viral proteins (U.S. Pat. Nos. 6,034,298; 6,136,320; 5,914,123 and 5,484,719(TGEV and hepatitis B); Mason et al, (1998) supra; Wigdorovitz, supra; Kapusta, et al, supra; McGarvey, P. B., J. Hammond, M. M. Dienelt, D. C. Hooper, Z. F. Fu, B. Dietzschold, H. Koprowski, and F. H. Michaels. 1995. Expression of the rabies virus glycoprotein in transgenic tomatoes. Biotechnology 13:1484-1487; Thanavala, Y., Y.-F. Yang, P. Lyons, H. S. Mason, and C. J. Arntzen. 1995. Immunogenicity of transgenic plant-derived hepatitis B surface antigen. Proc. Natl. Acad. Sci. U.S.A 92:3358-3361) and subunits of bacterial toxins (Arakawa, T., D. K. Chong, J. L. Merritt, W. H. Langridge. 1997. Expression of cholera toxin B subunit oligomers in transgenic potato plants. Transgenic Res. 6:403-413; Arakawa, T., J. Yu, and W. H. Langridge. 1999. Food plant-delivered cholera toxin B subunit for vaccination and immunotolerization. Adv. Exp. Med. Biol. 464:161-178; Haq, T. A., H. S. Mason, J. Clements, and C. J. Amtzen. 1995. Production of an orally immunogenic bacterial protein in transgenic plants: proof of concept of edible vaccines. Science 268:714-716). Animal and human immunization studies have demonstrated the effectiveness of many plant derived recombinant antigens in stimulating the immune system. The production of antigen-specific antibodies and protection against subsequent toxin or pathogen challenge demonstrates the feasibility of plant derived-antigens for immunologic use.

Some of the first edible vaccine technologies developed include transgenic potatoes expressing the E. coli heat-labile enterotoxin (LT-B), a Hepatitis B surface antigen (HbsAg); (Thanavala, Y., Y.-F. Yang, P. Lyons, H. S. Mason, and C. J. Arntzen. 1995. Immunogenicity of transgenic plant-derived hepatitis B surface antigen. Proc. Natl. Acad. Sci. U.S.A 92:3358-3361; Amtzen, C. J., D. M.-K. Lam. 2000. Vaccines expressed in plants. U.S. Pat. No. 6,136,320; Lam, D. M.-K., C. J. Amtzen, H. S. Mason. 2000. Vaccines expressed in plants. U.S. Pat. No. 6,034,298; Arntzen, C. J., D. M.-K. Lam. 1999. Vaccines expressed in plants. U.S. Pat. No. 5,914,123; Lam, D. M.-K., C. J. Amtzen. 1997. Anti-viral vaccines expressed in plants. U.S. Pat. No. 5,612,487; Lam, D. M., C. J. Amtzen. 1996. Vaccines produced and administered through edible plants. U.S. Pat. No. 5,484,719), and a Norwalk virus surface protein (Mason, H. S., J. M. Ball, J. J. Shi, X. Jiang, M. K. Estes, C. J. Amtzen. 1996. Expression of Norwalk virus capsid protein in transgenic tobacco and potato and its oral immunogenicity in mice. Proc. Natl. Acad. Sci. U.S.A. 93:5335-5340). In addition to human viral targets, two proteins specific for livestock viruses have also been expressed in plants and fed to animals to test for immune responses, VP1 protein for foot-and-mouth disease (Wigdorovitz, supra; Carillo, C., A. Wigdorovitz, J. C. Oliveros, P. I. Zamorano, A. M. Sadir, N. Gomez, J. Salinas, J. M. Escribano, M. V. Borca, 1998, Protective immune response to foot-and-mouth disease virus with VP1 expressed in transgenic plants. J. Virology 72:1688-1690) and Transmissable Gasteroenteritis Virus (Jilka, J. Immunogenicity of TGEV spike protein expressed in transgenic maize seed: preliminary swine trials. PCT/US01/01148).

One of the most promising aspects of edible vaccines is the ability of orally administered immunogens to stimulate a mucosal immune response (Ruedl, C. and H. Wolf. 1995. Features of oral immunization. Int. Arch. Allergy Immunol. 108:334-339). Mucosal surfaces, the linings of the respiratory, gastrointestinal, and urogenital tracts, play an important physical and chemical role in protecting the body from invading pathogens and harmful molecules. The mucosal immune system is distinct and independent of the systemic, or humoral, immune system, and is not effectively stimulated by parenteral administration of immunogens (Czerkinsky, C., A. M. Svennerholm, and J. Holmgren. 1993. Induction and assessment of immunity at enteromucosal surfaces in humans: implications for vaccine development. Clin. Infect. Dis. 16 Suppl 2:S106-S116). Rather, the mucosal immune system requires antigen presentation directly upon the mucosal surfaces (Jilka, J. Immunogenicity of TGEV spike protein expressed in transgenic maize seed: preliminary swine trials. WO 01/51080; Bailey, M. R. 2000. A model system for edible vaccination using recombinant avidin produced in corn seed. M.S. degree thesis, Texas A&M University). Since most invading pathogens first encounter one or more of the mucosal surfaces, stimulation of the mucosal immune system is often the best first defense against many transmissible diseases entering the body through oral, respiratory and urogenital routes (Holmgren, J., C. Czerkinsky, N. Lycke, and A. M. Svennerholm. 1994. Strategies for the induction of immune responses at mucosal surfaces making use of cholera toxin B subunit as immunogen, carrier, and adjuvant. Am. J. Trop. Med. Hyg. 50:42-54). It has been reported that mucosally administered SIV antigens can induce systemic and mucosal immune responses (Moldoveanu, Z., A. N. Vzorov, W. Q. Huang, J. Mestecky and R. W. Compans. 1999. Induction of immune responses to SIV antigens by mucosally administered vaccines. AIDS Research and Human Retroviruses 15:1469-1476; Yao, Q., V. Vuong, M. Li, and R. W. Compans. 2002. Intranasal immunization with SIV virus-like particles (VLPs) elicits systemic and mucosal immunity. Vaccine 20: 2537-2545).

Significant recent research has focused on the development of a vaccine against the human immunodeficiency virus (HIV). In 1981 the first cases of the acquired immune deficiency syndrome (AIDS) were recognized, and unrecognized cases were believed to have occurred for some years prior. In 1983 the agent responsible for AIDS, the human immunodeficiency virus (HIV) was isolated and identified. Two types of HIV have been identified, HIV-1, a highly virulent strain, is believed to be the cause of most AIDS cases in the world, whereas HIV-2 is found in West Africa and spreading into India. It is believed that the viruses were spread from other primates, such as the chimpanzee, to humans.

There now exists a pandemic of AIDS resulting in high human mortality and morbidity. The World Health Organization estimates 16.3 million people have died from AIDS and that 34.3 million people live with HIV infection. As a result, there has been considerable effort to study the disease and the virus which causes it, along with producing a vaccine to prevent its further spread.

HIV is an enveloped retrovirus, belonging to the group of retroviruses called lentiviruses. It is now believed the virus grows in the CD4 T-cells. The viron contains two copies of the RNA genome, and after infection and integration into the host cell chromosome, these are transcribed into DNA. These transcripts direct synthesis of viral proteins and also form the RNA genome of new particles. These new particles escape from the cell by budding from the plasma membrane.

Many recent studies have focused on the major envelope glycoprotein of HIV in the study of subunit vaccines against HIV and the related simian immunodeficiency virus, SIV. The protein gp160 and a processed form of this protein (gp120), for example, have been shown to possess many of the important epitopes for antibody recognition leading to virus neutralization. The simian equivalent of gp120 is gp130. These all serve the same purpose, of providing a surface protein. They are the dominant surface protein against which antibodies are raised.

HIV uses a complex of the two viral glycoproteins, gp120 and gp41 in the viral envelope. The gp120 binds to the CD4 molecule of the cell, and then binds to a coreceptor in the membrane of the host cell. The gp41 protein causes fusion of the cell membrane and viral envelope, and the virus then enters the host cell. (For a thorough discussion of HIV viral structure, see Immune Biology 5, The Immune System in Health and Disease. 2001. C. A. Janeway, P. Travers, M. Walport, M. Shlomchik, Garland Publishing, NY, N.Y., Chapt 11 “Failures of Host Defense Mechanisms” pp. 425-469.)

Vaccines produced against gp120 and gp160 have focused, most recently, on mucosal routes of immunization and have yielded variable yet promising results. (gp120: Bergmeier, L. A., E. A. Mitchell, G. Hall, M. P. Cranage, N. Cook, M. Dennis, and T. Lehner. 1998. Antibody-secreting cells specific for simian immunodeficiency virus antigens in lymphoid and mucosal tissues of immunized macaques. AIDS 12:1139-1147; Lu, X., H. Kiyono, D. Lu, S. Kawabata, J. Torten, S. Srinivasan, P. J. Dailey, J. R. McGhee, T. Lehner, and C. J. Miller. 1998. Targeted lymph-node immunization with whole inactivated simian immunodeficiency virus (SIV) or envelope and core subunit antigen vaccines does not reliably protect rhesus macaques from vaginal challenge with SIVmac251. AIDS 12: 1-10. gp160: (Ahmad, S., B. Lohman, M. Marthas, L. Giavedoni, Z. el Amad, N. L. Haigwood, C. J. Scandella, M. B. Gardner, P. A. Luciw, and T. Yilma. 1994. Reduced virus load in rhesus macaques immunized with recombinant gp160 and challenged with simian immunodeficiency virus. AIDS Res. Hum. Retroviruses 10: 195204; Moldoveanu, Z., A. N. Vzorov, W. Q. Huang, J. Mestecky, and R. W. Compans. 1999. Induction of immune responses to SIV antigens by mucosally administered vaccines. AIDS Res. Hum. Retroviruses 15:1469-1476) The gp160 protein includes gp120 and gp41. The gp120 protein extends upward from the viral membrane, whereas gp41 extends into the membrane. Production of an antibody response has been shown when mammals are exposed to the proteins; for, example the gp160 protein has been able to produce an antibody response in macaques and chimps (see e.g., Murphy-Corb et al., 1989. Science 246:1293-1297; Emini et al., 1989. J. Virol. 64:3674-3678; Chakrabarti, S. 1986. Nature 320:535; Hahn, B., 1985. Proc. Nat. Acad Sci USA 82:4813; U.S. Pat. No. 6,511,845), as has gp120 in mice (Chakrabarti et al. 1986 Nature 320(6062)535-7). The protein gp120 is a heavily glycosylated protein. This glycosylation acts equivalent to a protective jacket to the virus, and discourages antibody attack. However, there is a vulnerable area in the “variable region” of the V1, V2 and V3 loops. These loops protrude out and are less glycosylated. However they mutagenesize frequently, (3×10⁻⁵ per nucleotide base per cycle of replication), which leads to the generation of many variants of HIV within a single patient. Thus, the human immune system cannot mount an effective serum antibody response once an infection has taken hold. This allows time for the virus to enter the CD4 T-cells, where it becomes quiescent as proviral DNA.

Thus, there has also been work to develop a more stable version of the viral protein. A synthetic protein, which would in essence include all of the gp120 protein and half of the gp41 has been synthesized. This new protein is labeled gp140, and has been constructed to remove the normally occurring cleavage site (Binley, J. M., R. W. Sanders, A. Master, C. S. Cayanan, C. L. Wiley, L. Schiffner, B. Travis, S. Kuhmann, D. R. Burton, S. L. Hu, W. C. Olson, and J. P. Moore. 2002. Enhancing the proteolytic maturation of human immunodeficiency virus type 1 envelope glycoprotein. J. Virol. 76:2606-2616). This form of the surface protein has a more “open” architecture which may allow binding to antibodies that otherwise would not bind. It also is more stable and can be extracted in the trimeric form (Schulke, N., M. K. Vesanen, R. W. Sanders, P. Zhu, M. Lu, D. J. Anselma, A. R. Villa, P. W. H. I. Parren, J. M. Binley, K. H. Roux, P. J. Maddon, J. P. Moore and W. C. Olson. 2002. Oligomeric and conformational properties of a proteolytically mature, disulfide-stabilized human immunodeficiency virus type 1 gp140 envelope glycoprotein. J. Virol. 76:7760-7776).

One of the models which has been used in development of a vaccine is based on the simian immunodeficiency virus (SIV), which infects Rhesus macaques, and is closely related to HIV. Subunit vaccines have been made from gp120 and tested on chimpanzees.

The expression of gp120, gp140, or other subunits important to HIV infection, produced in recombinant plants, could offer several exciting benefits to SIV/HIV vaccine research. Large quantities of immunologically active recombinant antigen could be produced, very economically, for research or vaccine production. The immunogen could be produced in a safe, directly edible or easily purified form allowing for studies on the efficacy of edible, oral, or parenteral HIV vaccines. Multicomponent vaccines could easily be formulated from the seed of transgenic plant lines to generate an increased chance for successful virus neutralization, in a stand-alone vaccination strategy, as a booster, or in combination with other vaccines and vaccination routes. Attempts have been made to express portions of an HIV related protein in plant viruses, and using plant viruses to infect tobacco. Durrani et al. (Durrani, Z., T. L. McInerney, L. McLain, T. Jones, T. Bellaby, F. R. Brennan, N. J. Dimmock. 1998. Intranasal immunization with a plant virus expressing a peptide from HIV-1 gp41 stimulates better mucosal and systemic HIV-1 specific IgA and IgG than oral immunization. J. Immunol. Meth. 220:93-103) genetically engineered cowpea mosaic virus to contain a 21 amino acid sequence from HIV gp41 and the virus was then replicated in tobacco. Intranasal inoculation with that virus stimulated a mucosal and systemic IgA and IgG response against the peptide in mice. Tobacco plants inoculated with alfalfa mosaic virus particles carrying the V3 loop of HIV-1 produced inoculum for mice which then produced neutralizing antibodies against HIV-1 (Yusibov, V., A. Modelska, K. Steplewski, M. Agadjanyan, D. Weiner, D. C. Hooper, H. Koprowski. 1997. Antigens produced in plants by infection with chimeric plant viruses immunize against rabies virus and HIV-1. Proc. Natl. Acad. Sci. USA 94:5784-5788). Finally, Zhang et al. (Zhang, G., C. Leung, L. Murdin, B. Rovinski, K. A. White. 2000. In planta expression of HIV-1 p24 protein using an RNA plant virus-based expression vector. Molecular Biotechnol. 14:99-107) used the tomato bushy stunt virus as an expression vector to produce HIV p24 protein. Successful expression of HIV-related proteins in plants has not yet been achieved in monocotyledonous plants.

### SUMMARY OF THE INVENTION

The invention is the expression of HIV-related surface proteins in monocotyledonous plants. In a further preferred embodiment, they are expressed in graminae, and in a still further preferred embodiment are expressed in maize. The proteins may be extracted from the plant, or the plant tissue used in various applications. In one such application, the plant tissue can be orally administered to an animal. In a still further preferred embodiment the invention relates to expression of HIV-related proteins at levels such that commercial production in plants is practical. In a preferred embodiment such levels are at least about 0.05% total soluble protein and in a still further preferred embodiment are at least about 0.1% total soluble protein. In yet another embodiment, a biomass is created by expressing the proteins in a plurality of plants where at least some of the plants express the proteins, then harvesting the biomass.

### BRIEF DESCRIPTION OF THE DRAWINGS

FIG. 1 is the nucleotide sequence encoding SIVmac239 gp130 (SEQ ID NO: 1)

FIG. 2 is the amino acid sequence for SIVmac239 gp130 (SEQ ID NO: 2)

FIG. 3 is the nucleotide sequence for a maize Ubil promoter variant (SEQ ID NO: 3)

FIG. 4 shows a Western analysis of callus samples from five SVA and three SVB events

FIG. 5 shows a Western analysis of T₁SVA seed.

FIG. 6 is the nucleotide sequence encoding HIV gp120 (SEQ ID NO: 4)

FIG. 7 is the amino acid sequence for HIV gp120 (SEQ ID NO: 5)

FIG. 8 is the nucleotide sequence encoding HIV gp140 (SEQ ID NO: 6)

FIG. 9 is the amino acid for HIV gp140 (SEQ ID NO: 7)

FIG. 10 shows a Western analysis of HVA01 callus

### DETAILED DESCRIPTION OF THE INVENTION

This invention relates to the expression of HIV-related proteins in plants, and which express at high levels. At expression levels in excess of 0.05%, development of a commercial production system becomes possible. In order for expression of such proteins to be commercially viable, that is, the production costs of expressing the proteins in plants is exceeded by amounts and value of the end product, expression levels of at least about 0.05% should be met. Commercial production is still more practical and achievable at levels of at least about 0.1%, and most preferably at levels of at least about 0.5% or higher. This invention further relates to stable transformation of plants with such proteins. As used herein stable transformation refers to the transfer of a nucleic acid fragment into a genome of a host organism resulting in genetically stable inheritance.

Reitz Jr. et al. set forth sequences encoding envelope proteins of HIV-1 strains. Expression of a peptide of gp41 in a plant-derived virus, the cowpea mosaic virus has been shown (Durrani et al. supra); a coat protein of alfalfa mosaic virus used as a carrier molecule with the V3 loop of HIV-1 to infect tobacco plants (Ysibov et al., supra); and the tomato bushy stunt virus with the p24 protein used to infect tobacco and cucumber plant cells. (Zhang et al, supra). However, expression of gp120, gp130, gp140, gp160 and other SIV and HIV related proteins in monocots have not been demonstrated. Monocots are often preferred host plants, since they have been studied very extensively, can be adapted for higher levels of expression through plant breeding and other techniques, and have been shown to be capable of expressing heterologous proteins at high levels. With a large membrane-bound protein, as with the HIV-related proteins, codon optimization is necessary for optimal expression in plants, and in particular, in maize.

Genes which encode HIV-related proteins are available to one skilled in the art. See for example the extensive work of Robert Gallo, reflected in such U.S. patents as Franchini et al, U.S. Pat. No. 5,223,423, describing the genomic clone of HIV-2; Reitz Jr. et al., U.S. Pat. Nos. 5,420,030, 5,576,000 and 5,869,313, describing sequences encoding envelope proteins; Paolelth et al, U.S. Pat. No. 5,863,542, showing sequences for gp120; Berrada et al. (1995) J. Virol 69:6770-6778, Gao F. et al., (1996) J Virol 16511657, also Kessous-Elbaz, U.S. Pat. No. 5,850,001, Kierry et al, U.S. Pat. No. 5,169,763, showing gp160 encoding sequences and their use; Durrani et al, supra, showing a portion of the gp41 protein and Chada et al., (1993) J. Virol. 67:3409-3417, Respess et al., U.S. Pat. No. 6,333,195, showing sequences encoding gp120 and gp41; Ysibov, supra, showing V3 loop-encoding sequences; Sia et al., U.S. Pat. No. 6,395,714 showing sequences encoding gp140; and Zhang et al, supra, showing p24. This is an exemplary list of the numerous sequences known to those skilled in the art which can be employed in the present invention, and is meant to be illustrative. The methods available for putting together a gene for improved expression can differ in detail. However, the methods generally include the designing and synthesis of overlapping, complementary synthetic oligonucleotides which are annealed and ligated together and subjected to rounds of the polymerase chain reaction to yield a full length gene with convenient restriction enzyme sites for cloning. Oligonucleotide sequences can be chosen to maximize expression in the selected host by selection codons that are commonly used in that host and by avoiding potential messenger RNA destabilizing sequences.

Once the gene has been constructed it is placed into an expression vector by standard sub-cloning methods. The selection of an appropriate expression vector will depend upon the method of introducing the expression vector into host cells. A typical expression vector contains prokaryotic DNA elements coding for a bacterial replication origin and an antibiotic resistance gene to allow for the growth and selection of the expression vector in the bacterial host; a cloning site for insertion of an exogenous DNA sequence, which in this context would code for the protein of interest; eukaryotic DNA elements that control initiation of transcription of the exogenous gene, such as a promoter; and DNA elements that control the processing of transcripts, such as transcription termination/polyadenylation sequences. It also can contain such sequences as are needed for the eventual integration of the vector into the plant chromosome.

In a preferred embodiment, the expression vector also contains a gene encoding a selection marker which is functionally linked to a promoter that controls transcription initiation and a terminator that controls the termination of transcription. For a general description of plant expression vectors and reporter genes, see Gruber et al., “Vectors for Plant Transformation” in Methods of Plant Molecular Biology and Biotechnology 89-119 (CRC Press, 1993).

Promoter elements employed to control expression of the enzyme encoding gene and the selection gene, respectively, can be any plant-compatible promoter. These can be plant gene promoters, such as, for example, the ubiquitin promoter, the promoter for the small subunit of ribulose-1,5-bis-phosphate carboxylase, or promoters from the tumorinducing plasmids from Agrobacterium tumefaciens, such as the nopaline synthase and octopine synthase promoters, or viral promoters such as the cauliflower mosaic virus (CaMV) 19S and 35S promoters or the figwort mosaic virus 35S promoter. See Kay et al. (1987) Science 236:1299 and European patent application No. 0 342 926. See international application WO 91/19806 for a review of illustrative plant promoters suitably employed in the present invention. The range of available plant compatible promoters includes tissue specific and inducible promoters. In one embodiment of the present invention, the exogenous DNA is under the transcriptional control of a plant ubiquitin promoter variant. Plant ubiquitin promoters are well known in the art, as evidenced by European patent application no. 0 342 926.

Alternatively, a tissue specific promoter can be provided to direct transcription of the DNA preferentially to the seed. One such promoter is the globulin-1 promoter. This is the promoter of the maize globulin-1 gene, described by Belanger, F. C. and Kriz, A. L. (1991) Genetics 129:863-972. It also can be found as accession number L22344 in the GenBank database. Another example is the phaseolin promoter. See, Bustos et al. (1989) The Plant Cell Vol. 1, 839-853.

One option for use of a selective gene is a glufosinate-resistance encoding DNA and in an embodiment can be the phosphinothricin acetyl transferase (“PAT”) or maize optimized PAT gene (Jayne et al, U.S. Pat. No. 6,096,947) under the control of the CaMV 35S promoter. The gene confers resistance to bialaphos. See, Gordon-Kamm et al. (1990); Uchimiya et al., (1993) Bio/Technology 11:835, and Anzai et al., (1989) Mol. Gen. Gen. 219:492.

It may also be desirable to provide for inclusion of sequences to direct expression of the protein to a particular part of the cell. A variety of such sequences are known to those skilled in the art. For example, if it is preferred that expression be directed to the cell wall, this may be accomplished by use of a signal sequence and one such sequence is the barley alpha-amylase signal sequence. Rogers, (1985) J. Biol Chem 260, 3731-3738. Another example is the brazil nut protein signal sequence when used in canola or other dicots. Another alternative is to express the enzyme in the endoplasmic reticulum of the plant cell. This may be accomplished by use of a localization sequence, such as KDEL. This sequence contains the binding site for a receptor in the endoplasmic reticulum. Munro, S. and Pelham, H. R. B. (1987) Cell. 48:899-907.

Obviously, many variations on the promoters, selectable markers and other components of the construct are available to one skilled in the art.

In accordance with the present invention, a transgenic plant is produced that contains a DNA molecule, comprised of elements as described above, integrated into its genome so that the plant expresses a heterologous enzyme-encoding DNA sequence. In order to create such a transgenic plant, the expression vectors containing the gene can be introduced into protoplasts, into intact tissues, such as immature embryos and meristems, into callus cultures, or into isolated cells. Preferably, expression vectors are introduced into intact tissues. General methods of culturing plant tissues are provided, for example, by Miki et al., (1993) “Procedures for Introducing Foreign DNA into Plants” in Methods in Plant Molecular Biology and Biotechnology, Glick et al. (eds) pp. 67-68 (CRC Press 1993) and by Phillips et al., (1988) “Cell/Tissue Culture and In Vitro Manipulation” in Corn and Corn Improvement 3d Edit. Sprague et al. (eds) pp. 345-387 (American Soc. Of Agronomy 1988). The selectable marker incorporated in the DNA molecule allows for selection of transformants.

Methods for introducing expression vectors into plant tissue available to one skilled in the art are varied and will depend on the plant selected. Procedures for transforming a wide variety of plant species are well known and described throughout the literature. See, e.g., Miki et al., supra; Klein et al., (1992) Bio/Technology 10:268; and Weisinger et al., (1988) Ann. Rev. Genet. supra: 421-477. For example, the DNA construct may be introduced into the genomic DNA of the plant cell using techniques such as microprojectile-mediated delivery, Klein et al., (1987) Nature 327: 70-73.; electroporation, Fromm et al., (1985) Proc. Natl. Acad. Sci. 82: 5824; polyethylene glycol (PEG) precipitation, Paszkowski et al., (1984) Embo J. 3: 2717-2722; direct gene transfer, WO 85/01856 and EP No. 0 275 069; in vitro protoplast transformation, U.S. Pat. No. 4,684,611; and microinjection of plant cell protoplasts or embryogenic callus. Crossway, (1985) Mol. Gen. Genetics 202:179-185. Co-cultivation of plant tissue with Agrobacterium tumefaciens is another option, where the DNA constructs are placed into a binary vector system. Ishida et al., (1996) Nature Biotechnology 14, 745-750. The virulence functions of the Agrobacterium tumefaciens host will direct the insertion of the construct into the plant cell DNA when the bacteria infect the cell. See, for example Horsch et al., (1984) Science 233: 496-498, and Fraley et al. (1983) Proc. Natl. Acad. Sci. 80:4803.

Standard methods for transformation of canola are described by Moloney et al., (1989) Plant Cell Reports 8:238-242. Corn transformation is described by Fromm et al. (1990) Bio/Technology 8:833 and Gordon-Kamm et al., The Plant Cell 2:603. Agrobacterium is primarily used in dicots, but certain monocots such as maize can be transformed by Agrobacterium. U.S. Pat. No. 5,550,318. Rice transformation is described by Hiei et al., (1994) The Plant Journal 6(2), 271-282, Christou et al., (1991) Trends in Biotechnology 10:239. Wheat can be transformed by techniques similar to those used for transforming corn or rice. Sorghum transformation is described by Wan et al., (1994) Plant Physiolog. 104:37. Soybean transformation is described in a number of publications, including U.S. Pat. No. 5,015,580.

In one preferred method, the Agrobacterium transformation methods of Ishida supra and also described in U.S. Pat. No. 5,591,616, are generally followed, with modifications that allow the inventors to recover transformants from Hill maize. The Ishida method uses the A188 variety of maize that produces Type I callus in culture. In one preferred embodiment the Hill maize line is used which initiates Type II embryogenic callus in culture. While Ishida recommends selection on phosphinothricin when using the bar or PAT gene for selection, another preferred embodiment provides for use of bialaphos instead.

The bacterial strain used in the Ishida protocol is LBA4404 with the 40 kb super binary plasmid containing three vir loci from the hypervirulent A281 strain. The plasmid has resistance to tetracycline. The cloning vector cointegrates with the super binary plasmid. Since the cloning vector has an E. coli specific replication origin, it cannot survive in Agrobacterium without cointegrating with the super binary plasmid. Since the LBA4404 strain is not highly virulent, and has limited application without the super binary plasmid, the inventors have found in yet another embodiment that the EHA101 strain is preferred. It is a disarmed helper strain derived from the hypervirulent A281 strain. The cointegrated super binary/cloning vector from the LBA4404 parent is isolated and electroporated into EHA 101, selecting for spectinomycin resistance. The plasmid is isolated to assure that the EHA101 contains the plasmid.

Further, the Ishida protocol as described provides for growing fresh culture of the Agrobacterium on plates, scraping the bacteria from the plates, and resuspending in the co-culture medium as stated in the '616 patent for incubation with the maize embryos. This medium includes 4.3 g MS salts, 0.5 mg nicotinic acid, 0.5 mg pyridoxine hydrochloride, 1.0 ml thiamine hydrochloride, casamino acids, 1.5 mg 2,4-Dichlorophenoxyacetic Acid (2,4-D), 68.5 g sucrose and 36 g glucose, all at a pH of 5.8. In a further preferred method, the bacteria are grown overnight in a 1 ml culture, then a fresh 10 ml culture re-inoculated the next day when transformation is to occur. The bacteria grow into log phase, and are harvested at a density of no more than OD600=0.6 and preferably between 0.2 and 0.5. The bacteria are then centrifuged to remove the media and resuspended in the co-culture medium. Medium preferred for Hill is used. This medium is described in considerable detail by Armstrong, C. I. and Green C. E. “Establishment and maintenance of friable, embryogenic maize callus and involvement of L-proline” Planta (1985) 154:207-214. The resuspension medium is the same as that described above. All further Hill media are as described in Armstrong et al. The result is redifferentiation of the plant cells and regeneration into a plant. Redifferentiation is sometimes referred to as dedifferentiation, but the former term more accurately describes the process where the cell begins with a form and identity, is placed on a medium in which it loses that identity, and becomes “reprogrammed” to have a new identity. Thus the scutellum cells become embryogenic callus.

The levels of expression of the gene of interest can be enhanced by the stable maintenance of a protein encoding gene on a chromosome of the transgenic plant. Use of linked genes, with herbicide resistance in physical proximity to the enzyme encoding gene, would allow for maintaining selective pressure on the transgenic plant population and for those plants where the genes of interest are not lost.

With transgenic plants according to the present invention, protein can be produced in commercial quantities. Thus, the selection and propagation techniques described above yield a plurality of transgenic plants which are harvested in a conventional manner. The plant with the protein can be used in the processing, or the protein extracted. Protein extraction from biomass can be accomplished by known methods which are discussed, for example, by Heney and Orr, (1981) Anal. Biochem. 114: 92-96.

It is evident to one skilled in the art that there can be loss of material in any extraction method used. Thus, a minimum level of expression is required for the process to be economically feasible. For the relatively small number of transgenic plants that show higher levels of expression, a genetic map can be generated, via conventional RFLP and PCR analysis, which identifies the approximate chromosomal location of the integrated DNA molecule. For exemplary methodologies in this regard, see Glick and Thompson (1993), in Methods in Plant Molecular Biology and Biotechnology 269-84 (CRC Press 1993). Genetic mapping can be effected, first to identify DNA fragments which contain the integrated DNA and then to locate the integration site more precisely. This further analysis would consist primarily of DNA hybridizations, subcloning and sequencing. The information thus obtained would allow for the cloning of a corresponding DNA fragment from a plant not engineered with a heterologous enzyme encoding gene. (Here, “corresponding” refers to a DNA fragment that hybridizes under stringent conditions to the fragment containing the enzyme encoding gene).

One of skill will recognize that after the expression cassette is stably incorporated in transgenic plants and confirmed to be operable, it can be introduced into other plants by sexual crossing. Standard breeding techniques can be used, depending upon the species to be crossed.

Commercial production of HIV-related proteins in plants is thus made possible by the invention. By commercial production is meant the expression of the proteins in plants such that use of the plant host system is practical and economically feasible. By expressing the proteins at levels of at least about 0.05% total soluble protein of plant tissue, adequate amounts of protein are produced in the plants to make commercial production practical.

In one embodiment of the invention, a biomass is created by producing a plurality of plants by the methods described above, where at least some of the plants express the HIVrelated proteins. The biomass created is then harvested. The plants may be used as the source of the proteins, with all or part of the plant used as the protein source. In a preferred embodiment of the invention, seed is used as the source of the proteins. This is particularly preferred when a promoter preferentially expressing the proteins to the seed is used. Alternatively, the protein may be extracted by wet milling, dry milling or any one of numerous procedures available.

### EXAMPLE 1

### Expression of SIV Envelope Surface Proteins in Plants

Stable expression of SIV mac239 gp130 protein in maize seed is shown in the data below. Expression levels were as high as 0.5% of total soluble protein. At such levels of expression, elicitation of an immune response is expected when the material is fed to animals as part of a normal feeding regime (Jilka, J. Immunogenicity of TGEV spike protein expressed in transgenic maize seed: preliminary swine trials. WO 01/51080). The plant tissue can also be used to extract large amounts of this protein for use as a reagent.

### Materials and Methods

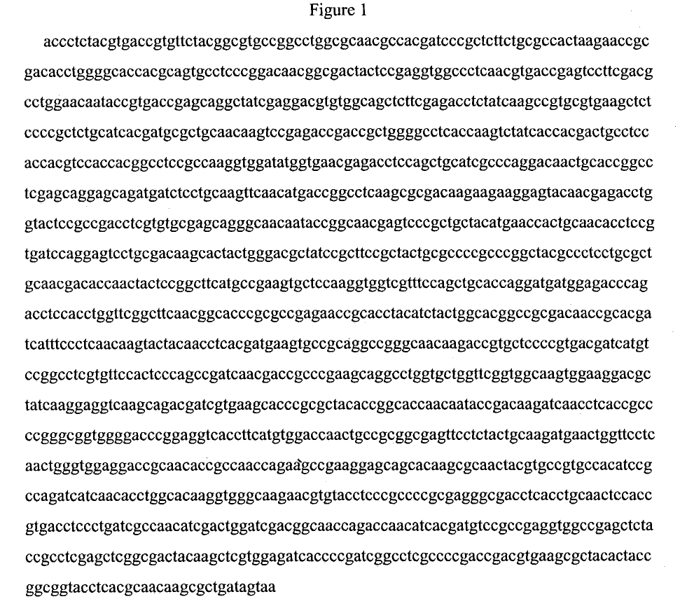

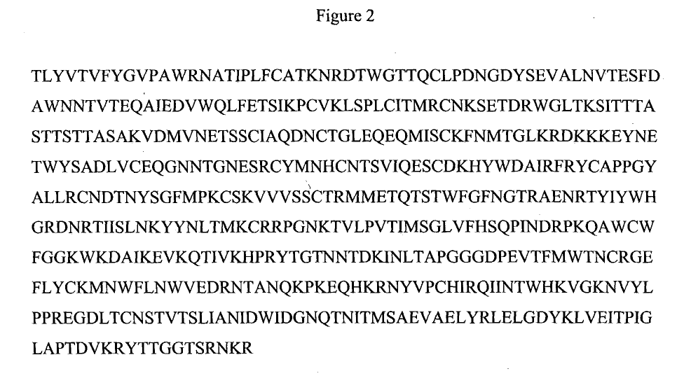

The SIV nucleotide sequence used in this example is set forth in FIG. 1 (SEQ ID NO: 1), having been synthesized for codon optimization in maize. The sequence ends in three stop codons (the start codon and BAASS-encoding sequence not included here). The correct mature cleaved SIVmac239 gp130 encoded is shown in FIG. 2 (SEQ ID NO: 2).

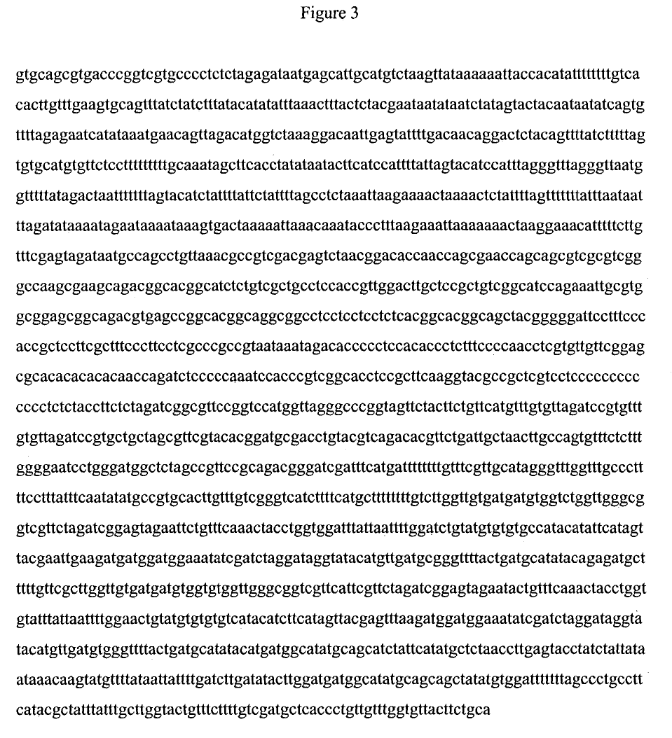

Immature embryos of corn (Zea mays L.) were isolated from greenhouse-grown ears at 9-13 days after pollination depending on embryo size, generally 1.5-2.0 mm long. The embryos were treated with A. tumefaciens containing the SIV mac239 gp130 gene with either a maize Ubi1 promoter with no heat shock elements (PGNpr4) see FIG. 3 (SEQ ID NO: 3; also PCT/US01/18689) or a maize globulinI promoter (Kriz, supra). Both constructs contained a barley α-amylase signal sequence (BAASS; Rogers et al. 1985 J. Biol Chem 260, 3731-3738), for targeting the protein into the cell wall (Streatfield, S. J., J. M. Jilka, E. E. Hood, D. D. Turner, M. R. Bailey, J. M. Mayor, S. L. Woodard, K. K. Beifuss, M. E. Horn, D. E. Delaney, I. R. Tizard, J. A. Howard. Plant-based vaccines: unique advantages. Vaccine 19:2742-2748), and both plant transcription units (PTUs) were terminated by the pinII terminator. (An et al., Plant Cell 1:115-122 (January 1989). Both constructs were attached to the 5′ end of a CAMV 35S-pat-35S PTU encoding resistance to the selective agent bialaphos. The maize vector constructed with PGNpr4 is designated PGN9065. The second SIV maize vector (PGN9066), designated for seed preferred expression was constructed using a fragment containing the maize globulin 1 promoter (Belanger et al, supra; GenBank accession L22344) in a three-way ligation with a fragment containing BAASS:SIVmac239 gp130 open reading frame plus the pinII terminator and the PGN8916 backbone which contains Ti plasmid and 35S:PAT:35S sequences (Hiei et al., supra).

The treated embryos were plated onto callus induction medium and incubated in the dark at 19° C. for four days. The embryos were then transferred to callus maintenance medium (CMM) and cultured in the dark at 28° C. They were transferred every two weeks to fresh CMM medium. The callused embryos ceased growing after about 2 weeks on bialaphos and eventually turned brown. Transgenic calli appeared as early as five weeks following treatment but the majority of events appeared at seven to nine weeks after treatment. The transgenic calli were easily spotted due to their white to pale yellow color, Type II callus phenotype, and rapid growth rate.

The transgenic events were grown for approximately four more weeks on bialaphos selection and then plated onto regeneration medium in the dark at 28° C. for somatic embryo production. The somatic embryos were removed after three weeks and plated onto germination medium in the light (20-30 μmoles·sec⁻¹m⁻²) at 25 embryos per plate at 28° C. The embryos germinated after 7-21 days and the To plantlets were moved into 25 mm×150 mm tubes containing 40 ml of minimal medium and left in the light as above for at least one week for further shoot and root development.

The plants were transferred into flats filled with equal parts of SunGro High Porosity (SunGro Horticulture Inc.) and Metro Mix 700 (Scott's-Sierra Horticultural Products Co.), covered with humidomes and placed in growth chambers for three to four weeks at 28° C. and 90 μmoles·sec⁻¹m⁻². Humidomes were removed after one week. Plants were transplanted into 2 gal pots filled with High Porosity potting media and 27 g of Sierra 17-612 slow release fertilizer mixed into the top media surface. Plants were moved to the greenhouse floor (27° C. and 195 μmoles·sec⁻¹ m⁻²). The To plants were pollinated with pollen from greenhouse-grown maize plants of elite germplasm.

Extraction of corn seed: Individual seeds were pulverized and homogenized with PBST (phosphate-buffered saline with 0.05% Tween-20™). Cell debris was removed by centrifugation. Total protein concentration was determined by the microtiter method (Bio-Rad, Richmond, Calif.) according to the method of Bradford (Bradford, M. M. 1976. A rapid and sesitive method for the quantitation of microgram quantities of protein utilizing the principal of protein-dye binding. Anal. Biochem. 72:248-254).

ELISA: Affinity-purified sheep anti SIV gp130 (cat # 6239) was obtained from Cliniqa, Inc. (Fallbrook, Calif.). Recombinant soluble human CD4 (cat #9759) was obtained from Protein Sciences Corp. (Meriden, Conn.). The following reagents were obtained through the NIH AIDS Research and Reference Program, Division of AIDS, NIAID, NIH: Recombinant SIVmac239 gp130 (cat.# 2322) from the DAIDS, NIAID (Hill, C. M., Deng, H., Unutmaz, D., Kewalramani, V. N., Bastiani, L., Gorny, M. K., Zolla-Pazner, S., Littman, D. R. 1997. Envelope glycoproteins from human immunodeficiency virus types 1 and 2 and simian immunodeficiency virus can use human CCR5 as a coreceptor for viral entry and make direct CD4-dependent interactions with this chemokine receptor. J. Virol 71:6296-6304); Rabbit anti-CD4 (T4-4, cat.# 806) from Dr. Raymond K. Sweet (Willey, R. L., Maldarelli, F., Martin, M. A., Strebel, K., 1992. Human immunodeficiency virus type 1 Vpu protein induces rapid degradation of CD4. J. Virol 66:7193-7200). This method is a modification of the ELISA for gp120-sCD4 described in the paper by J. P. Moore (Moore, J. P. 1990. Simple methods for monitoring HIV-1 and HIV-2 gp120 binding to soluble CD4 by enzyme-linked immunosorbent assay: HIV-2 has a 25-fold lower affinity than HIV-1 for soluble CD4. AIDS 4:297-305).

Dilutions of SIVmac239 gp130 standard (final concentration in assay 0.0034-0.067 ng/l) were incubated with 0.5 μg/ml CD4 in a buffer consisting of 0.2% Carnation Follow-up™ formula in PBST and 0.05 μg/μl non-transformed corn extract in polypropylene microtiter plates at 28° C. with constant shaking at 250 RPM for 1 h. Similarly, sample corn extracts were diluted to a final concentration of 0.05 μg/μl in 0.2% Carnation Follow-up™ formula in PBST and incubated with 0.5 μg/ml CD4. After preincubation, the samples and standards were transferred to Nunc Maxisorp plates (VWR, West Chester, Pa.) pre-adsorbed overnight with anti-SIV gp130 sheep antibody and incubated at 37° C. Complexes of gp130-CD4 bound to the plate were detected using anti-CD4 rabbit polyclonal antiserum and anti-rabbit alkaline phosphatase conjugate (Jackson Immunoresearch, West Grove, Pa.) followed by detection of colored product formation upon incubation with p-nitrophenyl phosphate (Sigma, St. Louis, Mo.).

Western analysis: Samples and standards were separated by SDS-PAGE under reducing and denaturing conditions using Novex 4-20% acrylamide gels (Invitrogen, Carlsbad, Calif.). Gels were subsequently blotted to Immobilon P PVDF (Millipore, Bedford, Mass.) and blocked with 5% non-fat dried milk in TBST (Tris-buffered saline with 0.05% Tween-20™). Blots were incubated with affinity purified sheep anti-SIV gp130 and detected with anti-sheep peroxidase (Jackson Immunoresearch, West Grove, Pa.) and the ECLTM substrate system (Amersham Pharmacia Biotech, Piscataway, N.J.).

### Expression of SIV Surface Proteins

Eleven stable transgenic maize events were recovered from the PGN9065 material (SVA) and 80 T₁ ears were harvested from 10 of these events. Sixteen stable events were recovered from the PGN9066 material (SVB). Thirteen of these events resulted in 127 ears of T₁ seed. These events resulted from a cross of the To plants with SP122, a StiffStalk-type elite germplasm. Crossing the Hi-II events with elite germplasm, in particular Stiff Stalk germplasm can increase event recovery. (See U.S. Ser. No. 10/349,392, to be published; Horn, Michael E.; Harkey, Robin L.; Vinas, Amanda K.; Drees, Carol F.; Barker, Donna K.; and Lane, Jeffrey R., “Use of Hill-Elite Hybrids in Agrobacteriumbased Transformation of Maize” In Vitro Cell. Dev. Biol.-Plant. (In press)). Stiff Stalk inbreds have been available since at least about the 1950s and are derived from the Iowa Stiff Stalk synthetic population. Sprague, G. F. “Early testing of inbred lines of maize” J. Amer. Soc. Agron. (1946)38:108-117; for examples see PI accession no. 550481 and discussion of Stiff Stalk germplasm at U.S. Pat. Nos. 5,706,603; 6,252,148; 5,245,975; 6,344,599; 5,134,074; and Neuhausen, S. “A survey of Iowa Stiff Stalk parents derived inbreds and BSS(HT)C5 using RFLP analysis” MNL (1989)63:110-111.

When analyzed using the indirect sandwich ELISA protocol described in the Materials and Methods, gp130 protein expression levels as high as 0.08% TSP for the SVA07 seed and as high as 0.022% TSP for the SVB07 material were observed (Table 1). Table 1 shows T₁ seed analysis of SVA and SVB seed. All To plants were derived from Hill.

TABLE 1

| | | Expression Level, | Number of seed |
|---|---|---|---|
| Construct | Event | high T₁seed % TSP | analyzed |
| SVA | 01 | 0 | 12 |
| | 02 | <0.0068 | 12 |
| PGNpr4: | 03 | 0.017 | 30 |
| BAASS:gp | 04 | 0.02 | 12 |
| 130 | 05 | 0.0086 | 6 |
| | 06 | 0.017 | 54 |
| | 07 | 0.078 | 42 |
| | 08 | 0 | 12 |
| | 09 | <0.0068 | 12 |
| | 10 | 0.0074 | 12 |
| SVB | 01 | 0.0068 | 12 |
| | 02 | 0 | 6 |
| PGNpr2: | 05 | <0.0068 | 12 |
| BAASS:gp | 07 | 0.022 | 36 |
| 130 | 08 | 0 | 12 |
| | 09 | <0.0068 | 12 |
| | 10 | 0.011 | 24 |
| | 12 | 0 | 12 |
| | 14 | <0.0068 | 12 |
| | 15 | 0 | 10 |

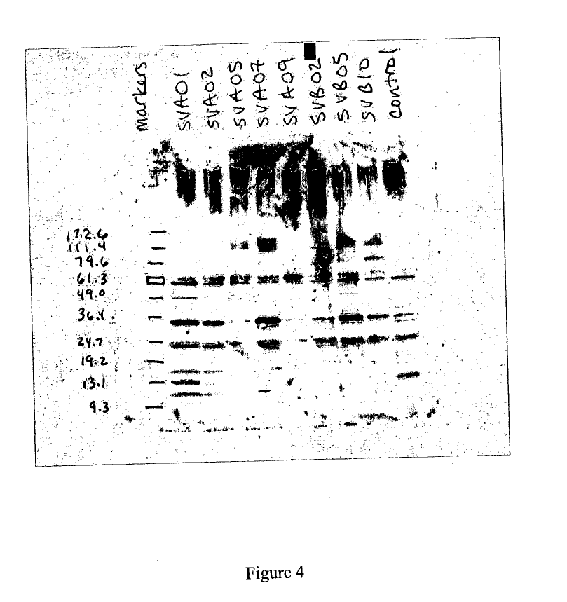

Western analyses of SVA and SVB callus showed novel immunospecific bands at 100-115 kDa, which corresponds approximately to native glycosylated gp130, and 58-60 kDa, which approximately agrees with the expected MW from the predicted amino acid sequence (FIG. 4).

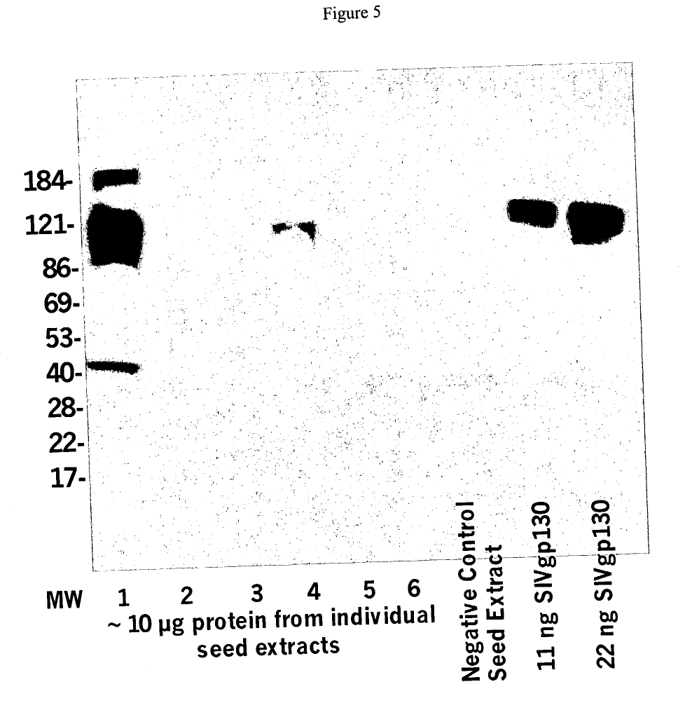

Western blot analyses of SVA T₁ seed is shown in FIG. 5. Estimating expression levels from the standard lanes, SVA07 shows approximately 0.2-0.3% TSP. The underestimation of SIV gp130 using the functional ELISA may be a result of poor binding of SIV gp130 protein to CD4 protein. These data show that the SIV gp130 protein is being expressed at levels that allow for extraction and purification for reagent purposes.

The expression level is also high enough to elicit an immune response in animals when fed to them as part of a reasonable and normal diet. This result is expected because of earlier studies with other viral subunit proteins (Arntzen, et al., supra; Jilka, J. Immunogenicity of TGEV spike protein expressed in transgenic maize seed: preliminary swine trials. WO 01/51080). Feeding corn-derived E. coli heat-labile enterotoxin subunit B (LT-B) to mice induced a strong mucosal and systemic immune response (Streatfield et al., supra). In fact, the LT-B delivered in corn induced a greater anti-LT-B specific mucosal IgA response than pure LT-B (Streatfield, et al, supra). This has also demonstrated this with a TGEV protein orally fed to swine (Jilka, et al supra). The results clearly demonstrate that an SIV surface protein, mac239 gp130, can be expressed in transgenic corn seed. Moreover, protein expression is at levels that are adequate to be used either to induce an immune response when fed, or as a reagent when extracted and purified.

It is believed that the difference in size between control gp130 (from whole virus) and corn-derived gp130 is a result of differences in glycosylation patterns between transgenic proteins expressed in animal cells and in plant cells. Plant cells are known to glycosylate proteins to a greater or lesser extent than the same proteins found in animal cells (Chargelegue, D., N. D. Vine, C. J. van Dolleweerd, P. M. W. Drake, J. K.-C. Ma. 2000. A murine monoclonal antibody produced in transgenic plants with plant-specific glycans is not immunogenic in mice. Transgenic Res. 9:187-194). This difference is chiefly due to the inability of plants to manufacture sialic acid. However, the difference in glycosylation pattern does not appear to alter the protein's immunogenic properties.

### EXAMPLE 2

### Expression of HIV Envelope Glycoprotreins in Plants

The SIV surface protein gp130 has been successfully expressed in plants at high levels. The same procedures set forth above were used to introduce the HIV equivalent into plants.

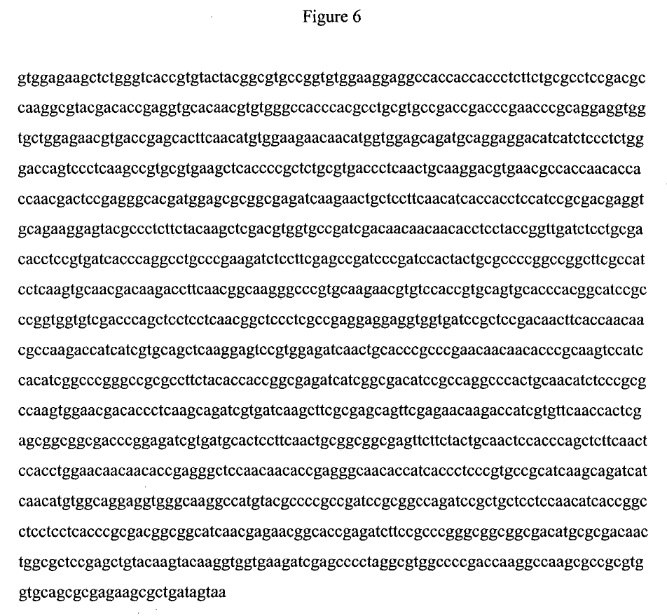

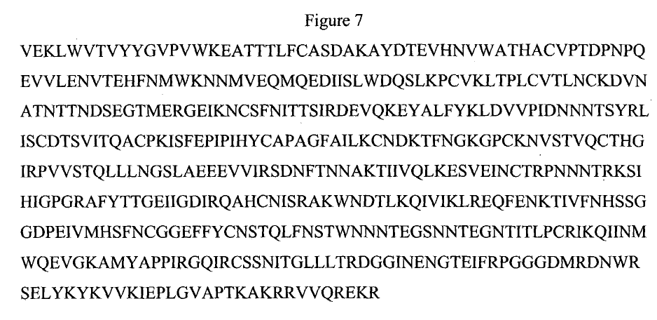

A synthetic version of an HIV gp120 segment of the env gene (GenBank accession U63632) was constructed in which codons were changed to reflect optimal codon usage in corn and to eliminate any potential message destabilizing sequences, see FIG. 6 (SEQ ID NO: 4). The amino acid sequence is shown in FIG. 7 (SEQ ID NO: 5). In addition, directly 5′, and in-frame with the HIV sequence, the construct contained an initiator methionine followed by a maize codon optimized barley alpha-amylase signal sequence (BAASS, GenBank accession K02637, Rogers, 1985 supra). To create a maize expression vector to direct constitutive expression, a three-way ligation was performed using a DNA fragment containing a ubiquitin promoter variant (PGNpr4), a fragment containing the HIV gp120 open reading frame, and the backbone of PGN8916, which contains the PinII terminator, beginning with a Pac I restriction site, along with Ti and 35S:PAT:35S sequences (Hiei et. al., 1994, supra). Sequence analysis confirmed that no errors were introduced.

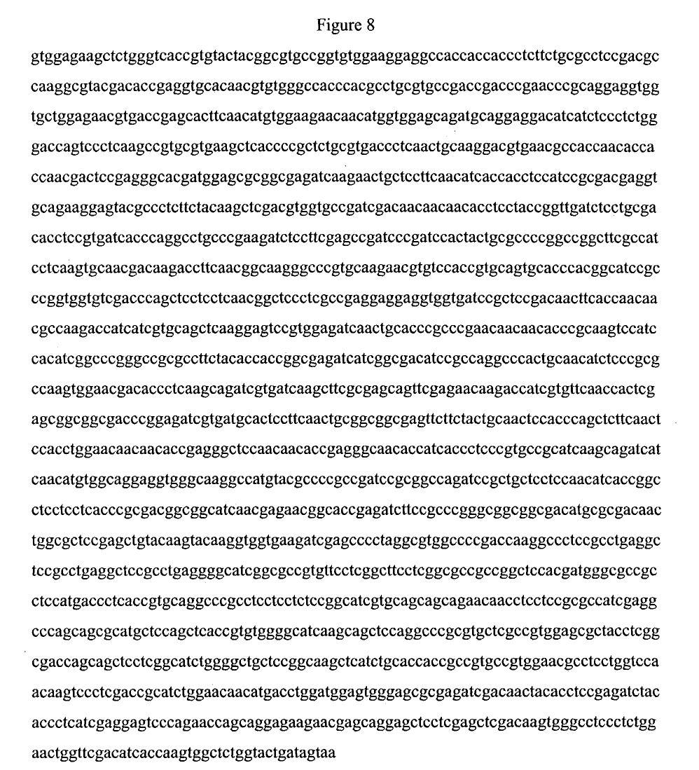

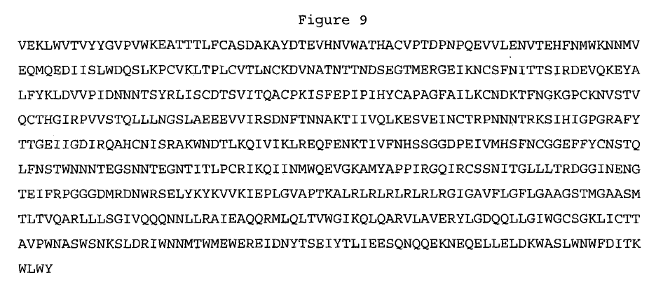

A synthetic version of an HIV gp140 segment of the env gene, designated gp140unc, (GenBank accession U63632) was assembled in which codons were changed to reflect optimal codon usage in corn, see FIG. 8 (SEQ ID NO: 6). The amino acid sequence is shown in FIG. 9 (SEQ ID NO: 7). This construct was built using a maize codon optimized synthetic gp120 construct (described above) in a ligation reaction with sequence shared with that of gp140 to utilize a restriction site for in-frame addition to the 3′ end of gp120 through to the C-terminus of gp140. The synthetic HIV sequence had also included sequence encoding an L/R hexamer to replace a putative furin cleavage site at amino acids 464 through 475 (amino acid number as in SEQ ID NO: 7), which may block cleavage in vivo, hence the designation gp140unc. In addition, directly 5′, and inframe with the HIV sequence, the synthetic construct contained an initiator methionine followed by a maize codon optimized barley alpha-amylase signal sequence (BAASS, GenBank accession K02637, Rogers, 1985 supra). To create a maize expression vector to direct constitutive expression, a three-way ligation was performed using a DNA fragment containing a ubiquitin promoter variant (PGNpr4), a fragment containing the HIV gp140unc open reading frame, and the backbone of PGN8916, which contains the PinII terminator, beginning with a PacI restriction site, along with Ti and 35S:PAT:35S sequences. The final construct was sequenced, which confirmed that no errors were introduced during cloning.

Anti-HIV Western analysis: Samples and standards were separated by SDS-PAGE under reducing and denaturing conditions using Novex 4-20% acrylamide gels (Invitrogen, Carlsbad, Calif.). Gels were subsequently blotted to Immobilon P PVDF (Millipore, Bedford, Mass.) and blocked with 5% non-fat dried milk in TBST (Tris-buffered saline with 0.05% Tween-20™). Blots were incubated with affinity purified sheep anti-HIV-1_{JR-FL}gp120 antibody (clinica, #6205) and detected with anti-sheep peroxidase conjugate (Jackson Immunoresearch, West Grove, Pa.) and the ECL+PluS™ substrate system (Amersham Pharmacia Biotech, Piscataway, N.J.).

### Expression of HIV Surface Proteins

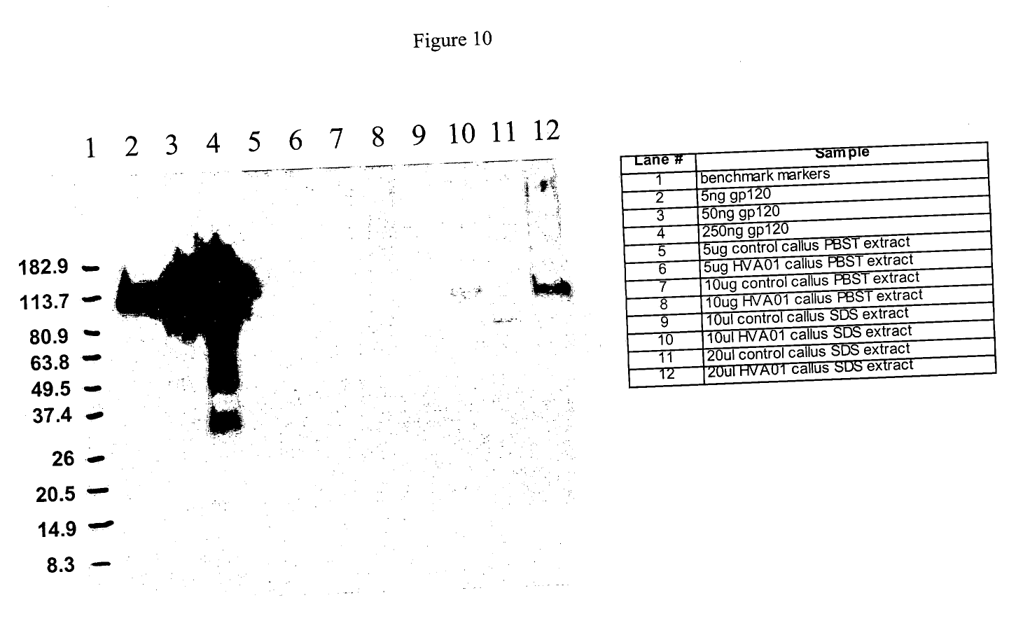

A single stable transgenic maize event from maize tissue transformed with HVA was harvested and extracted with PBST containing Complete protease inhibitor tabs (Roche, #1836153), and then extracted a second time with 1×SDS gel loading buffer without reducing agent. FIG. 10 shows an immunoblot analysis of HVA callus using an anti-HIV-1_{JR-FL}gp120 antibody. Western analyses of extracts prepared from HIV callus showed a novel immunospecific band at ˜110 kDa, which corresponds approximately to native HIV-1_{JR-FL}gp120 (FIG. 10). Estimating expression from the standard lanes indicates HIV-1_{JR-FL}gp120 in HVA01 is at least 0.05% of total soluble protein.

Feeding trials using the plant produced protein material will be undertaken using mice and simians. Both humoral and mucosal immune responses are expected.

What is claimed is:

1. A monocotyledonous plant expressing a simian or human immunodeficiency virus surface protein gene.

2. The plant of claim 1 wherein the plant is maize.

3. The plant of claim 1 wherein the surface protein gene is selected from the group consisting essentially of gp120, gp130, gp160, gp140 or gp41.

4. A monocotyledonous plant expressing a simian or human immunodeficiency virus surface protein selected from the group consisting essentially of gp120, gp130, gp160, gp140 or gp41.

5. The plant of claim 4 wherein the plant is maize.

6. A monocotyledonous plant stably expressing a simian or human immunodeficiency virus surface protein.

7. The plant of claim 6, the protein selected from the group consisting essentially of gp120, gp130, gp160, gp140 and gp41.

8. Seed of the plant of claim 6.

9. The plant of claim 1 wherein the surface protein is expressed in the plant at levels of at least about 0.1% total soluble protein.

10. The plant of claim 1 wherein the surface protein is expressed in the plant at levels of at least about 0.5% total soluble protein

11. The plant of claim 4 wherein the surface protein is expressed in the plant at levels of at least about 0.1% total soluble protein

12. The plant of claim 4 wherein the surface protein is expressed in the plant at levels of at least about 0.5% total soluble protein

13. The claim of claim 6 wherein the surface protein is expressed in the plant at levels of at least about 0.1% total soluble protein

14. The plant of claim 6 wherein the surface protein is expressed in the plant at levels of at least about 0.5% total soluble protein

15. A monocotyledonous plant stably expressing a simian or HIV surface protein at levels of at least about 0.1% total soluble protein.

16. The plant of claim 15 wherein the surface protein expresses at levels of at least about 0.5% total soluble protein.

18. Plant cells of a monocotyledonous plant expressing a simian or HIV surface protein gene.

19. Plant cells of claim 18 wherein the plant is a maize plant.

20. Plant cells of claim 18 wherein the surface protein gene is selected from the group consisting essentially of gp120, gp130, gp160, gp140 or gp41.

21. A method of producing simian or human immunodeficiency virus surface proteins in commercial quantities, comprising providing biomass from a plurality of monocotyledonous plants, of which at least certain plants contain a nucleotide molecule comprised of a heterologous nucleotide sequence coding for the surface proteins, wherein the nucleotide sequence is operably linked to a promoter to effect expression of the surface proteins by the certain plants such that the surface proteins are produced at levels of about 0.01% or higher of soluble protein in the certain plants.

22. The method of claim 21 further comprising extracting the proteins from the plants.

23. A method of producing an antibody response in an animal comprising administering to the animal a composition comprising monocotyledonous plant material containing HIV surface proteins comprising at least about 0.05% of the total soluble protein of the plant material.

24. The method of claim 23 wherein the HIV surface proteins comprise at least about 0.1% of the total soluble protein of the plant material.
